(19) United States
(12) Patent Application Publication
(10) Pub. No.: US 2012/0123124 A1
(43) Pub. Date: May 17, 2012

(54) Manufacturing process for Tadalafil from racemic or L-tryptophan

(75) Inventor: Milan Soukup, Sarasota, FL (US)

(73) Assignee: Drug Process Licensing Associates, LLC, Dallas, TX (US)

(21) Appl. No.: 13/066,684

(22) Filed: Apr. 22, 2011

(51) Int. Cl. C07D 471/04

(52) U.S. Cl. 546/85

(57) ABSTRACT

The present invention relates to a novel manufacturing process of pharmaceutically active compound of formula I, having (6R,12aR)-configuration, used for treatment of erectile dysfunction. Starting from racemic or L-tryptophan the invention describes preparation of an enantiomerically pure intermediate of formula II which is a known precursor in the synthesis of Tadalafil (formula I).

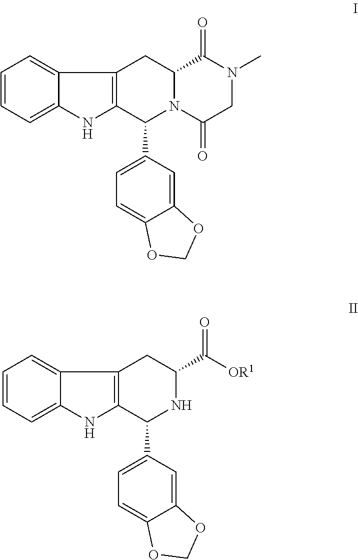

### BACKGROUND OF THE INVENTION

Tadalafil (compound of formula I), having the (6R,12aR)-configuration,

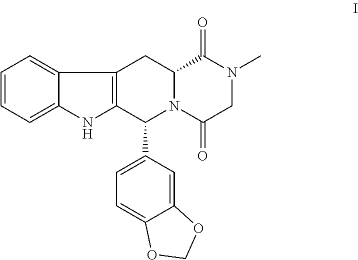

is a selective inhibitor of cGMP specific Type V phosphodiesterase (PDE5) and it is used for treatment of erectile dysfunction (Clalis®). The pharmacological activity of Tadalafil is specifically attributable to (6R,12aR)-enantiomer and many syntheses have been developed to prepare the enantiomerically pure compound. Since Tadalafil possesses at C(12a)-atom R-configuration, corresponding to configuration of D-tryptophan, all published syntheses have been using exclusively the significantly more expensive D-tryptophan as the starting material (U.S. Pat. No. 6,140,329, U.S. Pat. No. 6,127,542, Synlett 2004, 8, 1428, OPPI Briefs 2005, 37, No. 1, Tetrahedron Asymmetry 2008, 19, 435-442, ibid. 2009, 20, 2090, ibid. 2009, 20, 430, Synth. Commun. 2008, 38, 4265 and Europ. J. Org. Chem. 2010, 1711.

No synthesis of Tadalafil has ever been reported using either L- or rac.-tryptophan which are less expensive: L-tryptophan is less expensive because its industrial production is based on the fermentation of indole and serine using either wild-type or genetically modified bacteria. This conversion is catalyzed by the enzyme tryptophan synthase which cannot produce D-tryptophan. For the synthesis of Tadalafil the required, more expensive D-tryptophan has to be manufactured by a resolution of rac.-tryptophan prepared by chemical method. For cost efficient manufacture of Tadalafil there is a clear need for a new process in which the less expensive either L- or racemic tryptophan could be used.

### SUMMARY OF THE INVENTION

The present invention discloses a novel efficient process for the manufacture of enantiomerically pure Tadalafil from less expensive and readily available either L- or rac.-tryptophan as shown in Scheme 1:

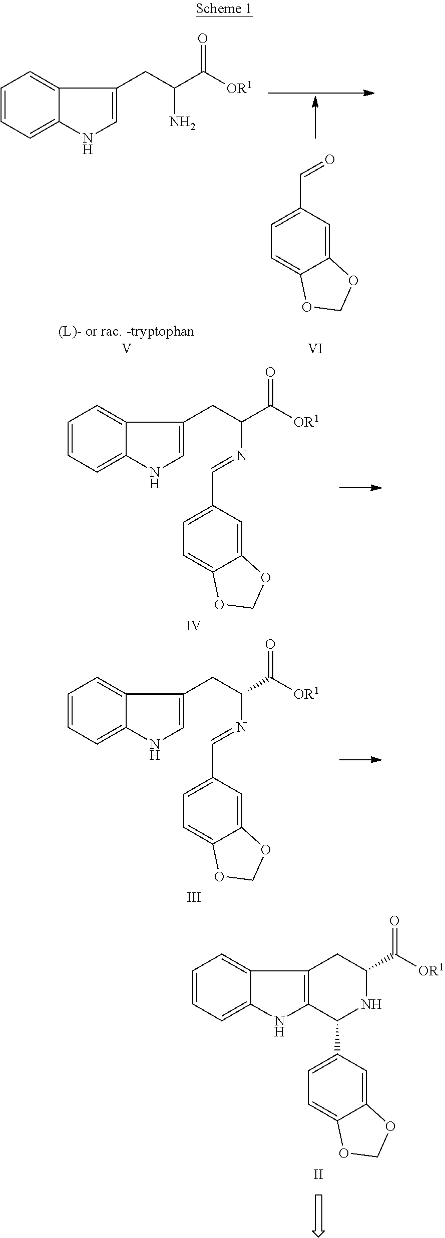

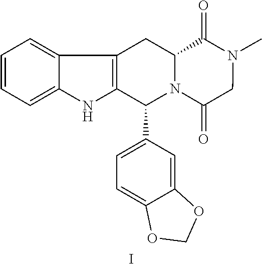

It has been unexpectedly found that the compound of formula II, which is an important intermediate in the synthesis of Tadalafil, having (1R,3R)-configuration can be efficiently prepared from inexpensive rac.- or L-tryptophan in high yield and high optical purity. Treatment of rac.- or L-tryptophan with piperonal of formula VI in the presence of suitable chiral acid (H—X) provides initially compound of formula IV which undergoes readily acid catalyzed epimerization at the carbon atom bearing the nitrogen function. If an appropriate solvent is used, in which the HX salt of compound of formula III is only limited soluble, crystallization induced asymmetric transformation converts finally all material of formula IV into the enantiomerically pure compound of formula III which undergoes stereo specific cyclization to enantiomerically pure intermediate of formula II. As shown in Tetrahedron Asymmetry 2008, 19, 435-442, this intermediate of formula II can be converted into Tadalafil in 2 steps.

### DETAILED DESCRIPTION OF THE INVENTION

The present invention claims a process (Scheme 1) for preparation of a compound of formula II, having (1R,3R)-configuration as given in the formula II,

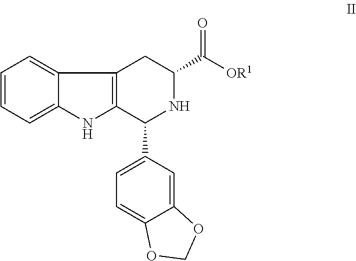

wherein R¹ represents hydrogen, alkyl, aryl, alkylaryl, arylalkyl, preferably hydrogen, methyl, ethyl and benzyl,
   from either L- or rac.-tryptophan of general formula V,

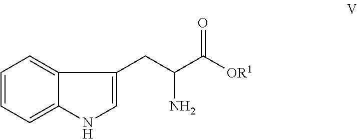

wherein R¹ is the same as defined for compound of formula II,
   by reacting with a compound of formula VI,

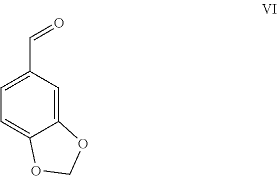

providing in situ compound of formula IV,

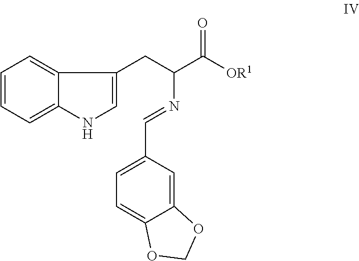

wherein R¹ is the same as defined for compound of formula II,
   which after addition of a suitable chiral acid H—X, preferably in stoichiometric amount, undergoes in suitable solvent under elevated temperature crystallization induced asymmetric transformation providing stereoselectivly enantiomerically pure compound of formula III,

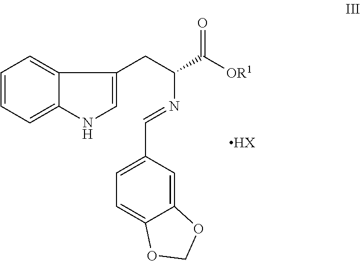

wherein R¹ is the same as defined for compound of formula II and HX is a suitable chiral acid,
   which spontaneously stereo selectively cyclizes to enantiomerically pure HX salt of the compound of formula II, which is collected from the precipitate and converted into an enantiomerically pure compound of formula II by treatment with suitable organic or inorganic base or using an ion-exchange resin.

Depending on the choice of starting material the compound of formula V can be present in the form as enantiomerically pure compound as (L)-tryptophan or as racemic tryptophan or as a mixture containing variable amount of both enantiomers.

As a resulting agent any chiral acid, as commonly used for resolution of nitrogen containing compounds, can be used. Preferably acids as (1R or 1S)-10-camphorsulfonic acid or (D or L)-tartaric acid or (D or L)-dibenzoyl tartaric acid, (1R or 1S)-3-bromocamphor-8-sulfonic acid, (+ or −)-1,1′-binaphtyl-2,2′-diyl-hydrogenphosphate itself or in a mixture with another aliphatic or aromatic carboxylic acid, preferably glacial acetic acid, can be used.

The chiral acid can be used in the amount of about 0.5 to 2 equivalents, preferably in stoichiometric amount.

The best results have been achieved specifically with (1R or 1S)-10-camphorsulfonic acid in a suitable solvent in which the compound of formula II is only limited soluble as e.g. acetonitrile, nitromethane, lower alcohols, preferably isopropanol, n-butanol, n-pentanol, THF, chlorinated hydrocarbons, preferably CHCl3, dichloroethylene, or dimethoxyethane. Also aromatic solvents as benzene, toluene, xylene or halogenated derivatives thereof, preferably toluene, can be used.

The reaction temperature for formation of the compound of formulas II, III and IV and for crystallization induced asymmetric transformation can be in the range of −10° C. until boiling temperature of the used solvent. Preferably reflux temperature in solvents as nitromethane or acetonitrile has been used.

A recrystallization from an appropriate solvent may further be useful to increase the diastereomeric excess (% ee) of the crystalline diastereomeric salt of formula II.

A small addition of lower alkyl carboxylic acids, as preferably acetic acid (up to one equivalent) or even addition of water can significantly promote the crystallization of the salt and increase the ee value.

In the further embodiment of the invention reaction of either L- or rac.-tryptophan of general formula V,

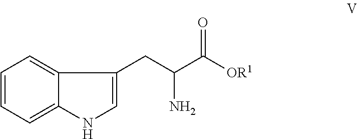

wherein R¹ represents hydrogen, alkyl, aryl, alkylaryl, arylalkyl, preferably hydrogen, methyl, ethyl and benzyl,
   with a compound of formula VI,

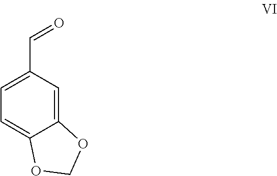

in the presence of a suitable chiral acid H—X, preferably in stoichiometric amount, under elevated temperature in a suitable solvent, followed by crystallization of the said mixture, collection of the desired diastereomeric salt from the precipitate and treatment of the salt with suitable organic or inorganic base, provides also the enantiomerically pure compound of formula II, having specifically the (1R,3R)-configuration.

In another embodiment of the invention a compound of general formula II, having the (1R,3R)-configuration as given in formula,

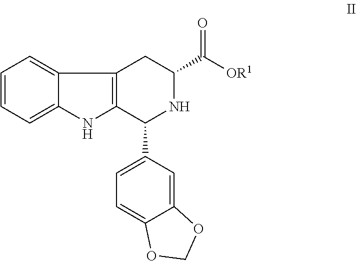

wherein R¹ represents hydrogen, alkyl, aryl, alkylaryl, arylalkyl, preferably hydrogen, methyl, ethyl and benzyl,
   can be also prepared from a compound of formula II, having any possible configuration at C(1)- and C(3)-chiral atoms, in the form as an enantiomerically pure compound or as a racemate or as a mixture of diastereomers,

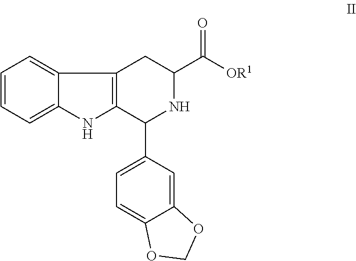

by adding a suitable chiral acid HX, preferably in stoichiometric amount, followed in a suitable solvent at elevated temperature crystallization induced asymmetric transformation, collection of the desired diastereomeric salt of compound of formula II from the precipitate and converting the salt into an enantiomerically pure compound of formula II by treatment with suitable organic or inorganic base or using an ion-exchange resin.

As a chiral acid preferably (1R or 1S)-10-camphorsulfonic acid or (1R or 1S)-3-bromocamphor-8-sulfonic acid in stoichiometric amount can be used. The reaction can be carried out preferably in boiling solvents as acetonitrile or nitromethane where the HX salt of the compound of formula II, having (1R,3R)-configuration, has only limited solubility. Under these conditions the starting material containing the compound of formula II, either in a form as enantiomerically pure compound or as racemate or diastereomeric mixture, undergoes crystallization induced asymmetric transformation providing enantiomerically pure HX salt of the compound of formula II, having specifically only (1R,3R)-configuration. This process is possible because at elevated temperature the chiral centers at C(1)- and C(3)-atoms in compound of formula II can be epimerized via its open structure intermediates of formulas IIc and IId as shown in Scheme 2. If an appropriate solvent is used, in which the HX salt of the compound of formula II, having (1R,3R)-configuration, is only limited soluble, crystallization induced asymmetric transformation converts finally all material into the enantiomerically pure compound of formula II specifically with (1R,3R)-configuration.

In addition dependent on a solvent a catalytic amount, preferably 5-10 mol.-%, of compound of formula VI can be beneficial for the asymmetric transformation.

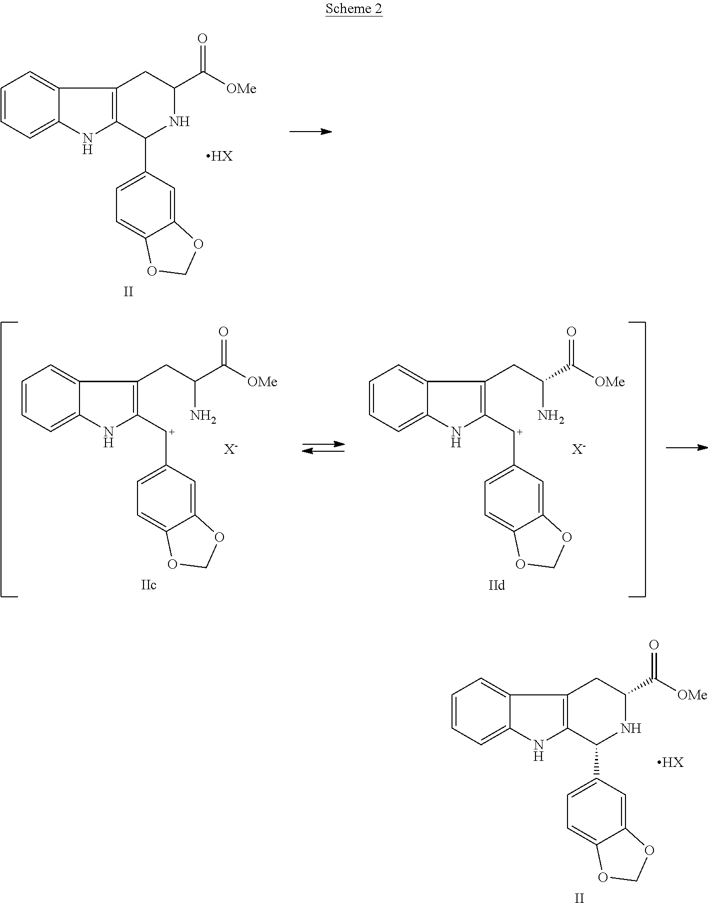

When referring to compounds described in the present invention, it is understood that references are also being made to salts thereof, preferably as H—X salts, wherein H—X is a suitable chiral acid.

In this invention a characteristic of protective group R¹ is that it can be removed readily (without the occurrence of undesired secondary reactions) for example by solvolysis, reduction, or alternatively under physiological conditions (as e.g. enzymatic cleavage or formation). Different protective group can be selected so that they can be removed selectively at different stages of the synthesis while other protective groups remain intact. The corresponding alternatives can be selected readily by a person skilled in the art from those given in the standard reference works mentioned in literature (as e.g. Mc Omie “Protective Groups in Organic Chemistry” or Green et al. “Protective Groups in Organic Synthesis”) or in the description or in the claims or the Examples.

For the purpose of this disclosure, a compound is considered to be “enantiomerically pure” if the content of one isomer is higher than 95%, preferably 99%.

The example are provided to illustrate particular aspects of the disclosure and do not limit the scope of the present invention as defined by the claims.

### EXAMPLES

Determination of optical purity was carried out with HPLC using chiral columns as Chiralcel OJ-H, Chiralpak AS-H or Chiralpak AD-H from Daicel Chem. Ind. In some cases the optical purity was also determined with NMR-Spectroscopy using chiral Eu-shift reagent. If not mentioned otherwise, all evaporations are performed under reduced pressure, preferably between 5-50 Torr, in some case even under high vacuum. The structure of final products, intermediates and starting materials is confirmed by standard analytical methods, e.g. spectroscopic characteristics as MS or NMR or IR. Abbreviations used are those conventional in the art.

### Preparation of (1R,3R)-1-(3,4-methylenedioxyphenyl)-2,3,4,9-tetrahydro-9H-pyrido[3,4-b]indole-3-carboxylic methyl ester (IIa) from L-tryptophan methyl ester (Va)

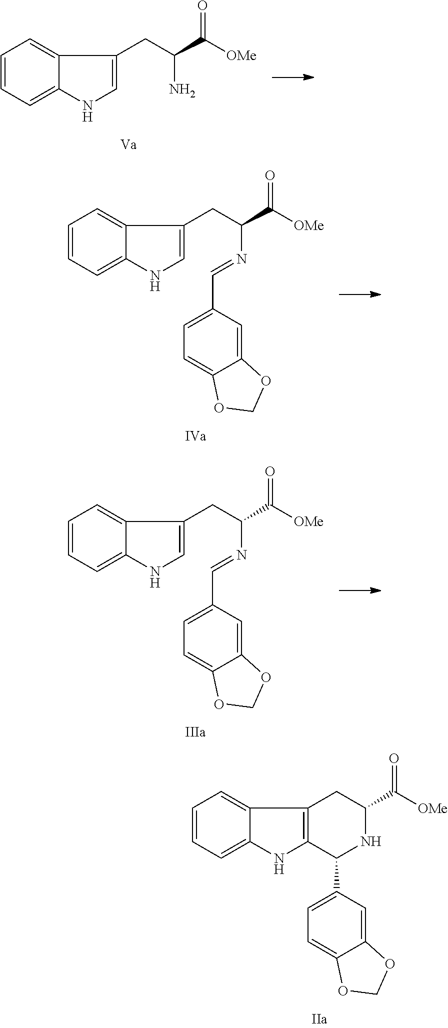

### Example 1

To a solution of piperonal (VI, 165 g), dissolved in dried acetonitrile (900 ml), under good stirring in inert atmosphere L-tryptophan methyl ester (Va, 220 g) and oven dried magnesium sulfate (500 g) were slowly added that the temperature stayed below 25° C. After complete addition the reaction slurry was stirred at rt over night, then filtered and the filter cake washed twice with acetonitrile (2×100 ml). To the filtrate (1R)-10-camphorsulfonic acid (232 g), dissolved in acetonitrile (400 ml), was slowly added, the mixture then seeded with crystals of the enantiomerically pure CSA-salt of compound (IIIa, 20 g), the slurry stirred over night and then heated under reflux for ca. 5 hrs (the reaction progress of the cyclization step was monitored by TLC). After slow cooling to 0° C. another portion of seeding crystals of the enantiomerically pure CSA-salt of the title compound (IIa, 20 g) was added and the slurry stirred over night. The precipitate was then collected by filtration, washed twice with cold acetonitrile (2×100 ml) and dried under vacuum to provide CSA salt of the title compound (IIa): 533 g (91.5% yield, 98% ee).

Crude CSA salt of IIa (533 g) was added upon an aqueous saturated NaHCO₃ solution (3000 ml) and methylenechloride (2000 ml) and shaken vigorously. The organic phase was separated, the aqueous phase washed twice with methylenechloride (2×300 ml), the combined organic phases dried over magnesium sulfate (100 g), filtered and the filtrate evaporated under reduced pressure to provide the title compound IIa: 301 g (86% yield, 98% ee).

For analytical purposes small sample of the crude product was purified by column chromatography on silica gel (eluens:hexane/ethyl acetate=8:1): Anal. calculated for C₂₀H₁₈N₂O₄: C, 68.56; H, 5.18; O N, 8.00; O 18.20. Found: C, 68.50; H, 5.22; N, 7.91; O 18.31. The analytical data of HCl salt of the title compound (IIa) was identical with analytical data as reported in Tetrahedron Asymmetry 2008, 19, 435-442.

### Preparation of (1R,3R)-1-(3,4-methylenedioxyphenyl)-2,3,4,9-tetrahydro-9H-pyrido[3,4-b]indole-3-carboxylic methyl ester (IIa) from L- or rac.-tryptophan methyl ester (Va or Vb)

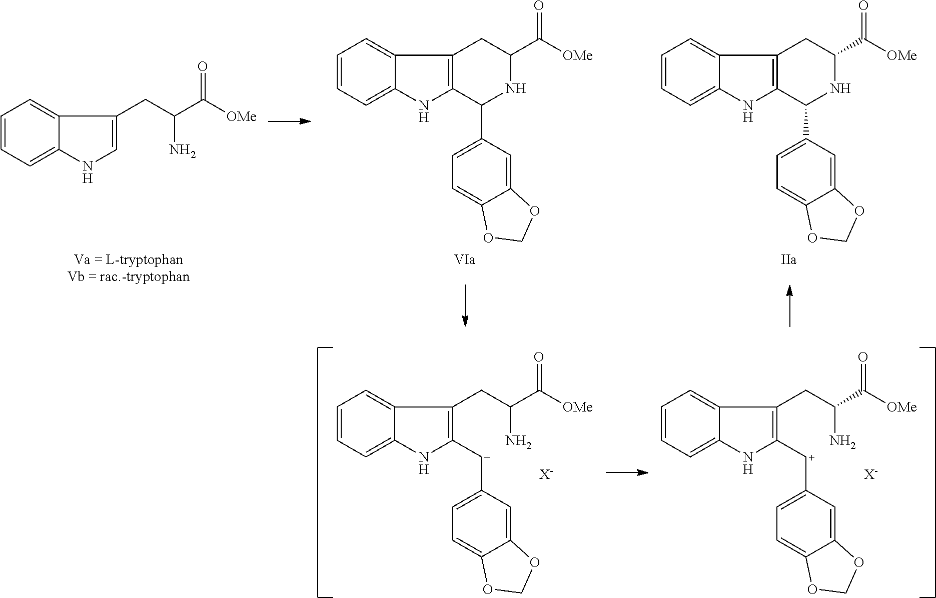

### Example 2

To a solution of piperonal (VI, 165 g), dissolved in dried acetonitrile (1000 ml), under good stirring in inert atmosphere rac.-tryptophan methyl ester (Vb, 220 g) and (1R)-10-camphorsulfonic acid (232 g) were slowly added that the reaction temperature stayed below 25° C. After complete addition the slurry was seeded with crystals of the enantiomerically pure CSA-salt of the title compound (IIa, 20 g), then stirred at rt over night, and afterwards heated under reflux for ca. 5 hrs (the reaction progress of the cyclization was monitored by TLC). After slow cooling to 0° C. second portion of seeding crystals (IIa) was added and the slurry stirred over night at 0° C. The precipitate was collected by filtration, washed twice with cold acetonitrile (2×100 ml) and dried under vacuum to provide CSA salt of the title compound (IIa): 501 g (86% yield, 97% ee).

### Example 3

To a solution of piperonal (VI, 175 g), dissolved in nitromethane (1100 ml), under good stirring in inert atmosphere rac.-tryptophan methyl ester (Vb, 220 g) and (1R)-10-camphorsulfonic acid (230 g), were slowly added that the temperature stayed below 30° C. After complete addition the slurry was seeded with crystals of the enantiomerically pure CSA-salt of the title compound (IIa, 20 g) and heated under reflux for ca. 5 hrs (the reaction progress of cyclization was monitored by TLC). After slow cooling to rt a second portion of seeding crystals (IIa) was added and the slurry stirred at 0° C. over night. The precipitate was collected by filtration, washed twice with cold nitromethane (2×100 ml) and dried under vacuum to provide CSA salt of the title compound (IIa) as pail yellow solid: 523 g (90% yield, 98.5% ee).

### Crystallization Induced Asymmetric Transformation Compound of Formula IIb into (1R,3R)-1-(3,4-methylenedioxyphenyl)-2,3,4,9-tetrahydro-9H-pyrido[3,4-b]indole-3-carboxylic methyl ester (IIa)

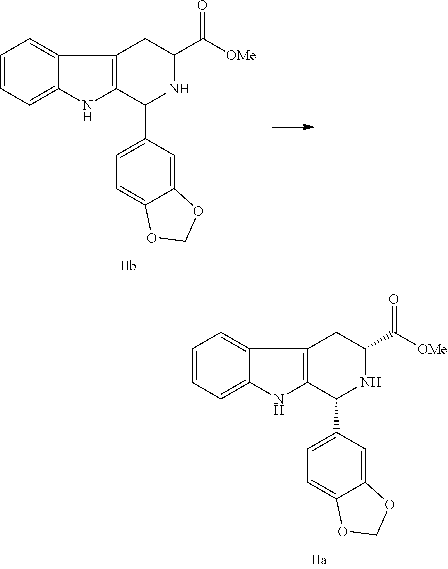

### Example 4

Under good stirring in inert atmosphere to a slurry of compound (IIb, 580 g) as a mixture of diastereomers in nitromethane (1100 ml), (1R)-10-camphorsulfonic acid (230 g) and piperonal (VI, 5 g) were added. The slurry was seeded with crystals of the enantiomerically pure CSA-salt of the title compound (IIa, 10 g) and then heated under reflux for ca. 8 hrs. After cooling to rt a second portion of seeding crystals (IIa) was added and the slurry stirred at 0° C. over night. The precipitate was collected by filtration, washed twice with cold nitromethane (2×100 ml) and dried under vacuum to provide CSA salt of the title compound (IIa) as pail yellow solid: 540 g (92% yield, 96% ee).

1. A process for preparation of a compound of formula II, having the (1R,3R)-configuration as given in the formula, 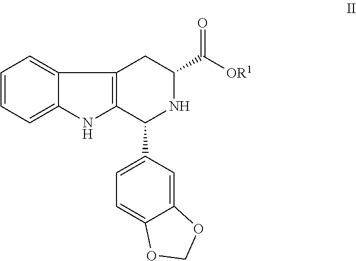
wherein R¹ represents hydrogen, alkyl, aryl, alkylaryl, arylalkyl, preferably hydrogen, methyl, ethyl and benzyl,
comprising following steps:
a) reaction of either L- or rac.-tryptophan of general formula V 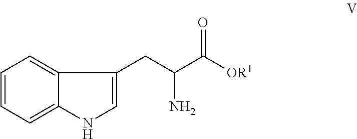
wherein R¹ is the same as defined for compound of formula II,
with a compound of formula VI, 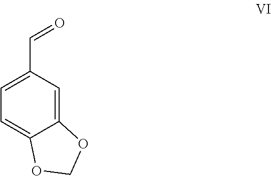
providing in situ a compound of formula IV, 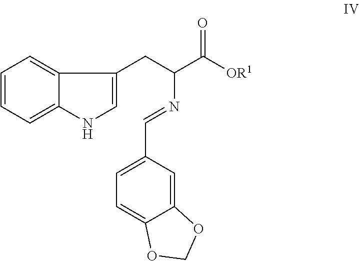
wherein R¹ is the same as defined for compound of formula II,
which after addition of a suitable chiral acid H—X, preferably in stoichiometric amount, undergoes in a suitable solvent, preferably acetonitrile or nitromethane, crystallization induced asymmetric transformation providing stereoselectivly an enantiomerically pure compound of formula III, 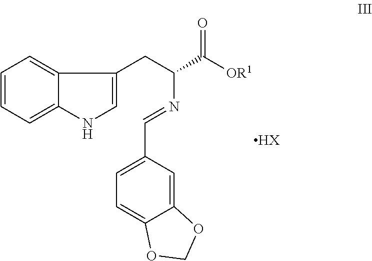
wherein R¹ is the same as defined for compound of formula II and HX is a suitable chiral acid,
which in situ undergoes stereo specific cyclization to enantiomerically pure HX salt of the compound of formula II,
b) collecting the diastereomeric salt of formula II from the precipitate and
c) converting the salt into an enantiomerically pure form of compound of formula II by treatment with suitable organic or inorganic base or using an ion-exchange resin.

2. A process for preparation of a compound of formula II, having the (1R,3R)-configuration as given in formula, 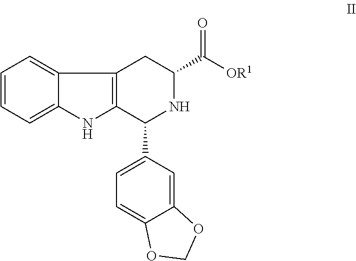
wherein R¹ represents hydrogen, alkyl, aryl, alkylaryl, arylalkyl, preferably hydrogen, methyl, ethyl and benzyl,
comprising following steps:
a) reaction of either L- or rac.-tryptophan of general formula V, 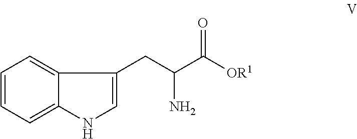
wherein R¹ is the same as defined for compound of formula II,
with a compound of formula VI, 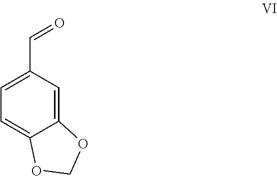
in the presence of a suitable chiral acid H—X, preferably in stoichiometric amount, in a suitable solvent, preferably acetonitrile or nitromethane, providing via stereo specific cyclization and crystallization induced asymmetric transformation the enantiomerically pure HX salt of the compound of formula II,
b) collecting the diastereomeric HX salt of compound of formula II from the precipitate and
c) converting the salt into an enantiomerically pure compound of formula II by treatment with suitable organic or inorganic base or using an ion-exchange resin.

3. A process for preparation of the HX salt of compound of formula II, having the (1R,3R)-configuration as given in formula, 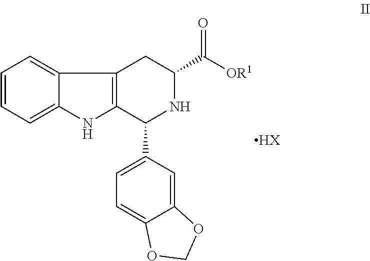
wherein R¹ represents hydrogen, alkyl, aryl, alkylaryl, arylalkyl, preferably hydrogen, methyl, ethyl and benzyl, and HX is a suitable chiral acid,
comprising crystallization induced asymmetric transformation of a compound of formula II, having any possible configuration at C(1)- and C(3)-chiral atoms, in a form as an enantiomerically pure compound or as a racemate or as a mixture of diastereomers, 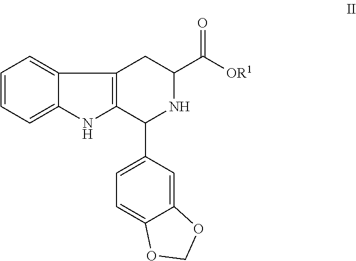
wherein R¹ is the same as defined for compound of formula II,
in the presence of a suitable chiral acid HX, preferably in stoichiometric amount, in suitable solvent, preferably acetonitrile or nitromethane, and
collecting the diastereomeric salt HX of the compound of formula II from the precipitate.

4. A process according to anyone of claims 1, 2 and 3, wherein the chiral acid HX is (1R or 1S)-10-camphorsulfonic acid or (D or L)-tartaric acid or (D or L)-dibenzoyl tartaric acid, (1R or 1S)-3-bromocamphor-8-sulfonic acid, (+ or −)-1,1′-binaphtyl-2,2′-diyl-hydrogenphosphate or (D or L)-mandelic acid, or alternatively, in a mixture with another aliphatic or aromatic carboxylic acid.

5. A process according to anyone of claims 1, 2 and 3, wherein the chiral acid HX is (1R or 1S)-10-camphorsulfonic acid.

6. A process according to anyone of claims 1, 2 and 3, wherein the chiral acid HX is (1R or 1S)-3-bromocamphor-8-sulfonic acid.

7. A process according to anyone of claims 1, 2 and 3, wherein R¹ is methyl.

8. A salt of the compound of formula II, having (1R,3R)-configuration as given in formula, 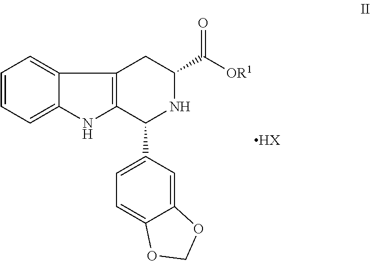
wherein R¹ is hydrogen, alkyl, aryl, alkylaryl, arylalkyl, preferably hydrogen, methyl, ethyl and benzyl, and
HX is (1R or 1S)-10-camphorsulfonic acid or (D or L)-tartaric acid or (D or L)-dibenzoyl tartaric acid, (1R or 1S)-3-bromocamphor-8-sulfonic acid in either enantiomerically enriched or enantiomerically pure form.
